(19) United States
(12) Patent Application Publication
(10) Pub. No.: US 2025/0269200 A1
(43) Pub. Date: Aug. 28, 2025

(54) ILLUMINATION DEVICE HAVING LIGHT DISTRIBUTION ELEMENT FOR IRRADIATION USING UV LIGHT AND TREATMENT SYSTEM FOR IRRADIATION USING UV LIGHT

(71) Applicant: SCHOTT AG, Mainz (DE)

(72) Inventors: Jens Ulrich THOMAS, Mainz (DE); Hubertus RUSSERT, Mainz (DE); Bernd SCHULTHEIS, Mainz (DE); Bernd HOPPE, Mainz (DE); Carolin HESSINGER, Mainz (DE); Yakup GÖNÜLLÜ, Mainz (DE)

(73) Assignee: SCHOTT AG, Mainz (DE)

(21) Appl. No.: 19/045,820

(22) Filed: Feb. 5, 2025

(30) Foreign Application Priority Data
Feb. 23, 2024 (DE) 10 2024 105 158.2

(51) Int. Cl. A61N 5/06; A61L 2/00; A61M 25/00; A61M 39/02

(52) U.S. Cl. CPC A61N 5/0624; A61L 2/0047; A61M 39/0247; A61L 2202/11; A61M 2025/0019; A61M 2039/0285; A61N 2005/063; A61N 2005/0632; A61N 2005/0661

(57) ABSTRACT

An illumination device having a light source which emits light at a wavelength of 180 nm to 360 nm, and a light distribution element having two opposing lateral surfaces is provided. The light distribution element includes a transparent or largely transparent material to the light coupled in. The light of the light source is coupled into the light distribution element and emerges from a lateral surface of the light distribution element which has structures for scattering the light coupled in, to deflect the light so that it emerges from a lateral surface. The light distribution element has a passage opening, which extends from one lateral surface of the light distribution element to the another lateral surface and is formed as a passage for a catheter. A device for sterilizing the skin, having the illumination device and a catheter is also disclosed.

### CROSS REFERENCE TO RELATED APPLICATIONS

This application claims the benefit of priority from German Patent Application No. 10 2024 105 158.2, filed Feb. 23, 2024, the disclosure of which is incorporated herein by reference.

### FIELD OF THE INVENTION

The invention relates in general to an illumination device, in particular for a medical treatment system, for irradiation using UV light. The illumination device comprises in this case at least one light source which emits UV light, as well as a light distribution element for generating a uniform distribution of the UV light within the predetermined application area. In particular, the invention relates to an illumination device for reducing the germ density on the skin or for disinfecting the skin.

### BACKGROUND OF THE INVENTION

Catheters are used in medicine in the context of diagnostic methods or therapeutic treatments. Catheters are understood as tubes or hoses of various diameters, using which hollow organs such as the bladder, stomach, intestine, or also blood vessels can be probed, emptied, filled, or flushed. In particular venous catheters have great importance in clinical practice. For this purpose, venous catheters, which are also referred to as central venous catheters or peripheral venous indwelling catheters, offer the possibility of supplying medications continuously or as needed to the patient over a relatively long period of time or, for example, taking blood for diagnostic purposes. The puncture of the veins through the skin represents an injury, however, and therefore the risk of infection by pathogens, which increases with the length of the dwell time of the catheter.

To reduce the risk of infection at the puncture point, attempts are therefore made to keep the number of pathogens, i.e. the number of germs, as low as possible in the area of the puncture point. For example, devices are known from the prior art which at least temporarily reduce the number of germs or pathogens on the skin of the patient in the area around the puncture point by irradiation of UV radiation.

Corresponding devices are known from DE 10 2009 015 088A. Devices of the type VisiLED UV ring light from Schott have a UV light source, using which the area around the puncture point can be irradiated using UV light. However, since the devices are relatively large, a continuous disinfection of the puncture point over the entire dwell time of the catheter is usually not possible or is at least not practical. Moreover, these devices are quite costly. A uniform and accurate irradiation of (skin) areas having a surface area in the range of 1 to 25 cm² also proves to be difficult. A further disadvantage in the use of corresponding devices is that if a spot UV source is used, only relatively small areas can be irradiated and in addition areas on the skin are shaded by the catheter and therefore experience no or only inadequate UV treatment. A uniform distribution of the UV light is technically demanding here due to the low wavelength, since most materials which are used for corresponding optical elements are not or are not sufficiently transparent to UV light having wavelengths below 300 nm.

### OBJECT OF THE INVENTION

One object of the present invention is therefore to provide an illumination device using which the number of germs on the skin, in particular at the puncture point of a catheter or in the adjoining areas, can be reduced or kept low easily and over hours, days, or even weeks by irradiation of UV light. For this purpose, the illumination device is in particular to have a structure which enables easy handling both during the introduction of the catheter by trained medical personnel and also as the illumination device remains on the patient during the dwell time of the catheter. A further object of the invention is to provide a device comprising a catheter and an illumination device for reducing the number of germs on the skin in the area of the puncture point.

The objects underlying the invention are already achieved by the subject matter of the independent claims. Advantageous embodiments and refinements are the subject matter of the dependent claims.

### SUMMARY OF THE INVENTION

One aspect of the invention relates to an illumination device for emitting UV light. The illumination device is suitable in particular to be used as part of a treatment system, a medical therapy system, or a diagnostic system and comprises at least one light source which emits light having a bactericidal effect, in particular light having a wavelength in the range of 180 to 360 nm. The light source is therefore a UV light source. Both conventional light sources such as mercury vapor lamps and also LEDs can be used as the UV light source. Furthermore, the illumination device comprises a disc-shaped light distribution element which emits the UV light uniformly over an area, wherein the UV light is scattered. The disc-shaped light distribution element has two opposing lateral surfaces and at least one surface between the two lateral surfaces. The surface which is located between the two opposing lateral surfaces and is at an angle, preferably at an angle of 90° to these lateral surfaces, is referred to in the scope of the disclosure as the end side or end face. The light distribution element comprises a material which is transparent or at least largely transparent for the light emitted by the light source. In particular, the material of the light distribution element has an absorption and/or scattering of less than 10% per centimeter, preferably of less than 1% per centimeter, for the light emitted by the light source for a wavelength in the range of 180 nm to 360 nm. Alternatively or additionally, the material of the light distribution element has a damping of less than −3 dB/cm, preferably of less than −1 dB/cm, for the light emitted by the light source. The light distribution element can consist of the UV-transparent material or comprise the transparent material. One embodiment provides that the light distribution element contains the UV-transparent material as the main component. Preferably, more than 50 wt. % or more than 70 wt. % of the light distribution element is a UV-transparent material. The UV-transparent material can also be used as a substrate which can be provided, for example, with one or more coatings. It has been shown that the UV-transparent material can both be amorphous and can also be crystalline or partially crystalline. According to one embodiment, quartz glass, preferably water-enriched quartz glass, is used as the UV-transparent material. According to another embodiment, crystalline CaF₂, crystalline MgF₂, or sapphire is used as the UV-transparent material.

The light emitted by the light source is coupled into the light distribution element, at least partially deflected by structures, and emerges from at least one of the two opposing lateral surfaces, preferably from one of the two opposing lateral surfaces. The light distribution element is therefore a diffuser element. The lateral surface from which the light is decoupled is also referred to in the scope of the disclosure as the decoupling lateral surface. The light distribution element has structures for scattering the UV light. These are preferably attached on or at one of the two lateral surfaces of the light distribution element. It has proven to be particularly advantageous if at least the lateral surface of the light distribution element through which the UV light is decoupled has structures for scattering the UV light.

The light distribution element has an opening, which extends from one lateral surface to the other opposing lateral surface of the light distribution element. The opening therefore forms a channel, which is open at both ends, through the light distribution element. The opening is preferably applied in a central area of the light distribution element. The opening is referred to in the meaning of the disclosure as a passage or passage opening. In particular, this opening is designed to function as a passage for a hose, in particular for a catheter, or a venous indwelling cannula. If the illumination device is used with a catheter, the decoupling lateral surface of the light distribution element is located on the side of the catheter tip. The UV light emerging from the decoupling lateral surface of the light distribution element is therefore incident on the skin in the area of the puncture point. The UV light is preferably emitted over the entire decoupling lateral surface of the light distribution element. Shading by the shadow of the catheter is minimized and a homogeneous irradiation is enabled by this arrangement.

In addition to the use of the illumination device in conjunction with catheters or similar therapy systems remaining in or on the body of a patient, such an illumination device having a light distribution element for distributing UV light is generally suitable for use in a medical treatment system, for example, if the application of UV light is to take place in particular for germ reduction or disinfection for the diagnosis or treatment or preparations for this purpose or also if the application of UV light is necessary, reasonable, or desirable before, during, and/or after a treatment. In order that the UV light emitted from the decoupling lateral surface of the light distribution element results in killing of germs and therefore a disinfectant or germicidal effect unfolds, a minimum light intensity of the UV light incident on the skin surface is necessary. At the same time, the intensity of the UV light incident on the skin cannot be excessively high, in order to avoid cell damage to the skin. According to one embodiment, the light intensity at a distance of 5 to 20 mm from the decoupling lateral surface of the light distribution element is therefore 1 to 50 μW/mm², preferably 2 to 20 μW/mm², and particularly preferably 5 to 15 μW/mm². Alternatively or additionally, the light emerging from the decoupling lateral surface has an integral light power of at least 1 mW, preferably of at least 2 mW. The integral light power is understood here as the energy at a distance of 5 to 20 mm to the decoupling lateral surface and integrated over the entire illumination area of the emitted light.

The corresponding light intensity is to be distributed as homogeneously as possible over the entire irradiated region or the entire area below the decoupling lateral surface. In this way, it is ensured that a disinfecting effect occurs in the entire irradiated region. The disinfectant or germicidal effect is preferably greatest close to the puncture point of the catheter here. The homogeneous intensity distribution is achieved in particular here in that the UV light is not only emitted in a point from above, but rather is emitted over a relatively large area due to the use of the light distribution element. Moreover, the emission takes place relatively close to the skin surface to be irradiated. The shading of the catheter is significantly reduced by this arrangement in relation to a point emission.

According to one embodiment, the irradiation intensity of the UV light decoupled from the light distribution element is therefore homogenized on an area which is arranged below the light distribution element so that, along a circular boundary line at a predetermined distance from the center point of the opening up to at most 2 cm from this center point, the ratio of the maximum of the irradiation intensity and the minimum of the irradiation intensity has a ratio of at most 3, preferably of at most 2, and the irradiation intensity has its maximum in a region which is at most 1.5 cm, preferably at most 1 cm from the center point of the opening.

One embodiment provides that the region of the lateral surface from which the UV light is decoupled has an area in the range of 1 to 25 cm², preferably in the range of 1 to 20 cm², particularly preferably in the range of 1 to 8 cm², and very particularly preferably in the range of 1 to 4 cm².

According to one embodiment, the light distribution element is circular or ellipsoidal. For a homogeneous light propagation, it can also be advantageous if the outer shape of the light distribution element is not completely circular. One refinement thus provides that the light distribution element has circular or ellipsoidal components, but does not form a complete circle or a complete ellipse. The light distribution element can thus in particular be D-shaped, i.e. circular with a missing circular segment.

Alternatively, the light distribution element is shaped as a polygonal disc, preferably as a polygon having at least 4, particularly preferably at least 5 corners. A circular light distribution can also be avoided in this way, at the same time, however, the light distribution element has a high degree of symmetry, so that an alignment of the light distribution element can be substantially neglected during the use of the light distribution element.

Light distribution elements having a maximum transverse dimension in the range of 1 to 8 cm, preferably in the range of 2 to 6 cm have proven to be particularly advantageous in this case. A further embodiment provides that the light distribution element has a maximum transverse dimension in the range of 1 to 4 cm, preferably in the range of 1.5 to 3 cm. The maximum transverse dimension is understood as a maximum distance between two edges or points on the edge of the same lateral surface, wherein the passage opening is not taken into consideration in the determination of this distance. In circular light distribution elements, the maximum transverse dimension corresponds to the circle diameter. In particular if the illumination device is used in conjunction with a catheter, such as a venous catheter or a comparably dimensioned catheter, the above-described dimensions have proven to be advantageous. Thus, on the one hand, a homogeneous irradiation of a sufficiently large area is ensured by the size of the light distribution element, while the dimensions are at the same time small enough for uncomplicated handling, for example if the illumination device remains on the catheter over its entire dwell time in the patient.

One embodiment provides that the light source emits UV light of a wavelength in the range of 180 nm to 250 nm, in particular in the range of 200 to 230 nm. According to another embodiment, the light source emits UV light at a wavelength in the range of 250 to 300 nm. At sufficient light intensity, a disinfecting or germicidal effect of the emitted light is ensured in particular in these wavelength ranges. At the same time, the penetration depth, for example into the skin, is relatively small at these wavelengths, which is advantageous with regard to the desired disinfection of the skin surface.

The light distribution element comprises a material which is transparent or largely transparent to the light emitted by the light source. Both amorphous and crystalline materials can be used in this case. According to one embodiment, the light distribution element comprises or consists of UV-transparent SiO₂, sapphire, CaF₂, or MgF₂. The use of hydrated SiO₂ has proven to be particularly advantageous. The SiO₂ has a particularly low absorption in the UV range due to the water content or the additional hydroxy groups, in particular also for low wavelengths.

The light distribution element functions as a diffuser. It has been shown that a particularly homogeneous distribution can be achieved if one of the two lateral surfaces of the light distribution element, at least in sections, has a roughness RMS in the range of 1 to 400 nm, preferably in the range of 10 to 200 nm, and particularly preferably in the range of 50 to 150 nm. One embodiment provides that the lateral surface from which the light is decoupled has a corresponding roughness. The lateral surface can have a uniform roughness in this case. The RMS roughness is the so-called root-mean-squared roughness (square of the mean) and is calculated from the mean of the deviation squares and corresponds to the ‘root-mean-square’ of the measured values over a measuring distance 1. These can be determined, for example, by optical methods such as white light interferometry or confocal microscopic methods.

Alternatively, a light distribution element is provided in which the lateral surface having increased roughness, preferably the decoupling lateral surface, has sections having different roughness. The roughness therefore differs locally in this embodiment. The roughness can have a gradient curve in this case. The RMS roughness can have, for example, a gradient of 0.2 nm to 150 nm in this case. According to one embodiment, the lateral surface having increased roughness has an RMS roughness in the range of 0.2 to 100 nm, preferably in the range of 0.3 to 80 nm. The corresponding lateral surface can therefore have both very smooth or even polished regions and also roughened regions, i.e. regions having a higher roughness. The roughness preferably increases with increasing distance from the point at which the UV light is coupled into the light distribution element. In this way, regions which are farther away from the coupling point have a higher decoupling rate than the less rough regions close to the coupling point. In the meaning of the disclosure, the coupling point is understood in particular as the region of the light distribution element at which the light emitted by the light source is coupled into the light distribution element. Due to this effect of different roughness or a gradient of the roughness, losses which occur as the light is conducted further within the light distribution element, for example, due to scattering and/or decoupling, are equalized and therefore a homogeneous emission behavior can be achieved over the entire decoupling lateral surface of the light distribution element.

A comparable effect can alternatively or additionally be achieved by microstructuring on one of the two lateral surfaces of the light distribution element, preferably on the decoupling lateral surface of the light distribution element. The corresponding microstructures can be formed by various methods. In particular methods such as laser ablation, laser structuring, or hot shaping processes (e.g., pressing, embossing), and also wet-chemical or dry-chemical etching processes possibly having preceding lithography processes or also preceding laser structuring processes or laser processes which permit the material of the substrate to be modified so that it can be structured or etched in a deliberate or predeterminable manner, are suitable for this purpose. Microstructuring of one of the two lateral surfaces by sandblasting is also possible. Such microstructuring can contribute, in addition to the above-mentioned roughness, to the light being able to be distributed more homogeneously coming from the coupling point. One embodiment provides that the microstructures are formed by a structured coating, for example by a structured silver coating. According to one embodiment of the invention, the light distribution element has one or more microstructures in locally bounded regions of the lateral surface, using which the local intensity of the decoupled light or the proportion of the decoupled light can be adjusted. This can be carried out by light scattering. Microstructures are not understood as restricted to structures in the micrometer range in the meaning of the invention, but also larger or rougher structures and in particular smaller or finer structures in the sub-micrometer or nanometer range.

According to one embodiment, one of the two lateral surfaces of the light distribution element has a lower roughness than the other lateral surface. In particular, the lateral surface having the lower roughness has a roughness RMS of less than 0.5 nm, preferably less than 0.2 nm, and particularly preferably less than 0.1 nm.

The roughness of the lateral surface opposite to the decoupling lateral surface is preferably lower than the roughness of the decoupling lateral surface. In this way, the most complete possible reflection of the light at this lateral surface is promoted. The most complete possible reflection or even a total reflection at this lateral surface is advantageous since therefore nearly all of the light coupled in is emitted through the decoupling lateral surface and no or only a very low intensity loss of the light coupled in takes place through the opposite lateral surface. This intensity loss can be minimized further if at least one of the lateral surfaces of the light distribution element has a coating which is highly reflective for the wavelength of the UV light used, preferably a reflective aluminum coating or silver coating. In principle, multilayer dielectric reflector layers could also be used if they are designed in the layered sequence in such a way that the typically very high reflectance is also designed over large reflection angle ranges (>45° in relation to the perpendicular to the lateral surface). In this case, both the decoupling lateral surface and the lateral surface opposite thereto can have a corresponding coating.

According to one embodiment, the light distribution element has a one-piece, preferably monolithically formed substrate made of a material transparent to the emitted light of the light source. This enables in particular the light distribution element to be provided by a production method having few manufacturing steps. The substrate can thus be molded in one step and then coated, for example. This is advantageous in particular with regard to cost-effective production methods and additionally enables a compact, space-saving design of the light distribution element. Alternatively, however, the light distribution element can also be constructed from a multipart substrate. In other words, the light distribution element can be formed from or in a disc of a substrate material or also can be formed from or using at least two discs of one or more substrate materials. In the case of a multipart embodiment, the light guide element optionally comprises spacers between the at least two discs, which are arranged at least in some parts or sections on or at the outer radius of the discs and can be designed, for example, as rings or ring segments. Furthermore, it is conceivable in the case of the multipart embodiment that at least the material on the decoupling side is transparent in the relevant wavelength range. The other materials can, but possibly do not have to have this property.

One embodiment provides that the light distribution element is designed as a circular disc having a maximum diameter in the range of 20 to 30 mm and a thickness in the range of 0.4 to 1.5 mm. In this embodiment, the opening for the passage of a hose or catheter is preferably arranged centrally in the light distribution element and has a diameter in the range of 2 to 6 mm. The opening is preferably made circular or elliptical.

The walls of the opening can extend parallel to the normal of the surface, the opening therefore extends substantially perpendicular from the surface of one lateral surface to the surface of the opposite lateral surface.

Embodiments are also possible in which the opening for the passage of a hose or catheter is formed as a slot and the slot extends from an outer edge region of the light distribution element into a central region. These embodiments do have the disadvantage of a lower homogeneity of the emitted light, but enable the illumination device to be added to or also removed from a catheter already applied to the patient. In one refinement, the light distribution element can be designed as multipart. In this case, one part of the light distribution element has the slot for the passage, which can be closed or covered after the passage of the catheter through the slot using a further part. A high homogeneity of the decoupled light can also be achieved in this refinement in this way.

During the placement of the illumination device on the patient, it is advantageous if the decoupling lateral surface has a spatial distance to the skin of the patient. One refinement of the invention therefore provides that the light distribution element is fixed in a circumferential holder, wherein at least one region of the holder protrudes beyond the decoupling lateral surface of the light distribution element. This region of the holder is used as the spacer between light distribution element and the skin of the patient. This region of the holder preferably has a height in the range of 5 to 15 mm. The holder preferably comprises an organic polymer, in particular a polysiloxane (such as a silicone) or a polycarbonate, wherein the organic polymer is to have sufficient UV stability.

According to one embodiment, the light distribution element has an opening having inclined walls. In this embodiment, the walls of the opening preferably extend at an angle in the range of 5 to 45° in relation to the surface normal. This is advantageous in particular when positioning the illumination device at points on the patient which are difficult to access and/or with catheters which are to be placed at a flat angle. Such an inclined wall of the opening moreover would have the double function of guiding the catheter at a defined angle in relation to the skin when the light distribution element is aligned parallel to the skin surface by the holder element.

One embodiment of the invention provides that the light of the light source is coupled via at least one light guide into the light distribution element. This embodiment offers the advantage that light source and light distribution element can be spatially decoupled or separated from one another in this way. In this way, the part of the illumination device which is attached directly to the patient can be designed in the most space-saving possible manner. It is also possible in this case to couple light of the light source into the light distribution element via multiple light guides, in order to thus achieve a more homogeneous coupling over the entire light distribution element. The light guide or guides can be spliced or adhesively bonded to the surface or in the or through the end face of the light distribution element. One embodiment provides that the light guide or guides is/are connected by a glass or plastic solder or a polymer-based adhesive to the light distribution element.

According to one refinement, the light of the light source is coupled via at least one light guide into the light distribution element, wherein the light guide has a numeric aperture NA>0.1, preferably greater than 0.2, at its distal end, which is connected to the light distribution element. In this way, the light cone of the coupling region is already spread or the light cone originating from the light guide is widened. The outgoing light cone of the distal light guide end can moreover be further enlarged, for example, by bevelling or roughening the light guide fiber.

For coupling in the light, the distal end of the light guide is connected according to one embodiment to the lateral surface of the light distribution element opposite to the decoupling lateral surface.

Alternatively, the light guide can be connected via an end side of the light distribution element. In a disc-shaped light distribution element, the end side is understood, for example, as the circumferential surface between the two lateral surfaces.

One refinement provides that the coupling in of the light takes place via an inclined surface of the light distribution element. The inclined surface can be formed in particular by a section of the end face of the light distribution element. The inclined surface at which the light is coupled in preferably has an angle a in relation to the lateral surfaces of the light distribution element in the range of 30 to 50°. The inclined coupling-in surface can moreover have a roughness RMS of >1 nm, preferably >2 nm, particularly preferably >10 nm, and very particularly preferably >50 nm.

According to one embodiment, the coupling in of the light takes place via the edge surface which forms the passage opening for a hose or catheter. Shadowing due to the hose or catheter can be avoided or at least reduced by this coupling in of the light with tangential components to the radius along this opening. This is advantageous in order to possibly also ensure in shaded regions that the intensity of the UV light is sufficiently high to ensure effective sterilization or disinfection in the shaded region.

Another embodiment provides that the light of the light source is coupled directly from the light source into a lateral surface of the light distribution element. Direct coupling is understood in particular to mean that the light emitted by the light source radiates into the light distribution element originating from the light source and is not conducted via a light guide to the light distribution element. The direct coupling preferably takes place through the lateral surface of the light distribution element opposite to the lateral surface from which the light is decoupled. The light source is firmly connected to this lateral surface. This can be carried out, for example, by a holder or an adhesive bond. UV LEDs are preferably used as light sources. The use of UV laser diodes is also conceivable. Particularly uniform coupling of the light over the entire light distribution element can take place if the illumination device has multiple light sources connected in a formfitting or materially-bonded manner to the lateral surface of the light distribution element.

A further aspect of the invention relates to a device for sterilization, comprising the illumination device according to the invention and a catheter, wherein the catheter is guided through the opening of the light distribution element so that the tip of the catheter or the end of the catheter, using which the catheter is introduced into the body of the patient, is located on the decoupling lateral surface of the light distribution element. The illumination device comprises a circumferential holder, which protrudes beyond the light distribution element at least on the decoupling lateral surface of the light distribution element. The holder therefore functions as the spacer between the decoupling lateral surface of the light distribution element and the skin of the patient.

### BRIEF DESCRIPTION OF THE DRAWINGS

The invention is described in more detail hereinafter on the basis of exemplary embodiments and FIGS. 1 to 20. In the figures:

FIG. 1 shows a side view of a schematic illustration of the illumination device according to a first exemplary embodiment,

FIG. 2 shows the exemplary embodiment shown in FIG. 1 in a top view,

FIG. 3 shows the schematic illustration of a second exemplary embodiment of the illumination device in a top view,

FIGS. 4 to 6 show schematic illustrations of the light distribution element of various embodiments in cross section,

FIG. 7 shows a schematic illustration of an embodiment of a multipart light distribution element in cross section,

FIG. 8 shows a schematic illustration of an embodiment of a one-piece light distribution element having inclined coupling surface in cross section,

FIG. 9 shows a schematic illustration of an embodiment of a light distribution element having inclined passage opening in cross section,

FIG. 10 shows a schematic illustration of an embodiment of a light distribution element having a decoupling lateral surface having locally differing roughness in a top view of the decoupling lateral surface,

FIG. 11 shows a schematic illustration of a first exemplary embodiment of a device for sterilization of the skin with coupling in via a lateral surface of the light distribution element in cross section,

FIG. 12 shows a schematic illustration of a second exemplary embodiment of a device for sterilization of the skin with coupling in via an end face of the light distribution element in cross section,

FIG. 13 shows a schematic illustration of a cross section of a third exemplary embodiment of a device for sterilization of the skin, in which the light sources are materially bonded to the light distribution element,

FIG. 14 shows the schematic illustration of the embodiment shown in FIG. 13 in a top view,

FIG. 15 shows a schematic illustration of the structure for determining the homogeneity of the emitted light,

FIG. 16 shows a schematic illustration of the diameters d0 and d1 for determining the homogeneity of the emitted light,

FIG. 17 shows a schematic illustration for ascertaining the minimum shaded region,

FIG. 18 shows a schematic illustration for ascertaining the maximum shaded region, and

FIGS. 19, 20 show schematic illustrations of two exemplary embodiments of an illumination device having D-shaped or polygonal light distribution elements.

### DETAILED DESCRIPTION OF THE INVENTION

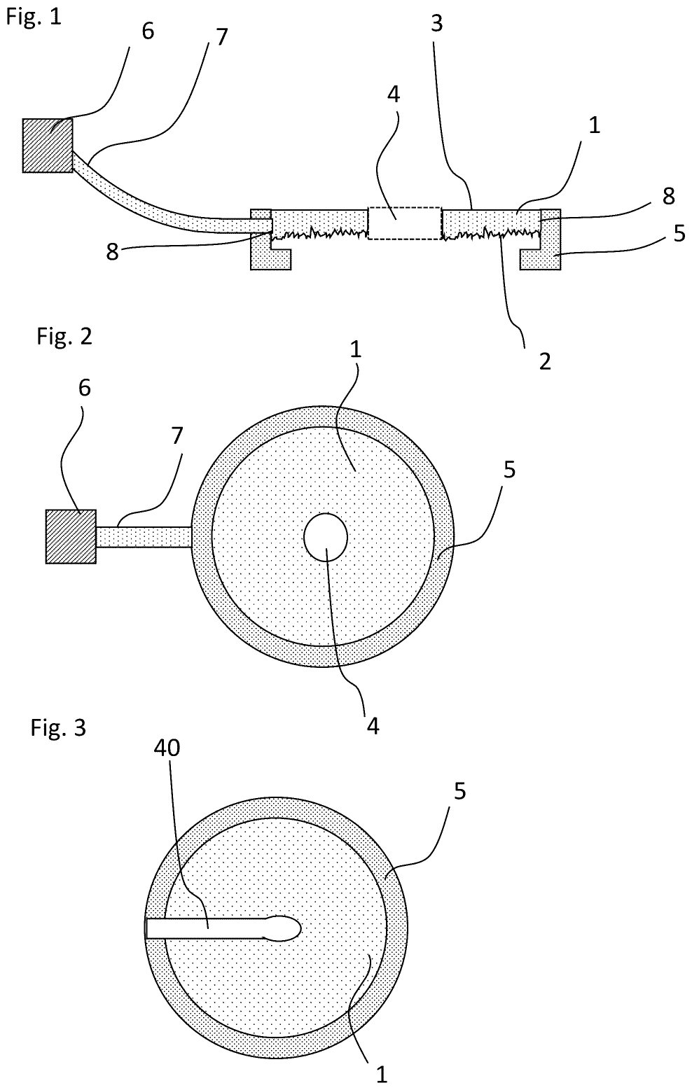

FIG. 1 shows a schematic cross section through an illumination device according to one exemplary embodiment. In this exemplary embodiment, the illumination device comprises a light source 6, which emits UV light at a wavelength in the range of 180 to 250 nm, and a light distribution element 1. The light distribution element 1 comprises a material which is transparent or at least largely transparent to the light emitted by the light source 6. In particular, the material of the light distribution element 1 has a damping for the light emitted by the light source 6 of less than −3 dB/cm. Light distribution element 1 and light source 6 are connected to one another via a light guide 7. Due to the use of a light guide 7, light source 6 and light distribution element 1 are spatially separated from one another. The length of the light guide 7 can be adapted to the respective conditions and is not shown to scale in the figures. The light guide 7 is in particular a quartz glass fiber. The light distribution element 1 is designed as a disc having the two lateral surfaces 3 and 2 and a circumferential end side 8. The coupling in of the UV light emitted by the light source 6 takes place on the end side 8 of the light distribution element 1. For this purpose, the light guide 7 is spliced, adhesively bonded, or fixed with, at, or in the end side 8 of the light distribution element 1 so that the distal fiber end is in flush contact with the end side. The lateral surface 2 of the light distribution element 1 is designed as the decoupling lateral surface and has a structuring in the form of a roughened surface. The decoupled light is scattered by the structuring applied to the lateral surface 2 so that the entire area below the light distribution element 1 is irradiated homogeneously or at least largely homogeneously. The light distribution element 1 has a passage opening 4 for the passage of a hose or catheter. The passage opening 4 extends through the material of the light distribution element 1 from the lateral surface 2 to the lateral surface 3 and therefore forms an open channel through the light distribution element 1. In the exemplary embodiment shown in FIG. 1, the opening 4 is arranged centrally in the light distribution element 1 and has vertical walls. The light distribution element 1 is held by a circumferential holder element 5, also referred to as a holder hereinafter. The holder 5 also extends below the decoupling lateral surface 2 and has an L-shaped profile in the exemplary embodiment shown. The holder 5 has an opening for the light guide 7 in the area of the coupling point.

FIG. 2 shows the above-described exemplary embodiment in a top view. It is clear that in this exemplary embodiment the light distribution element 1 is designed as a circular disc and the passage opening 4 is in the center point of this disc.

FIG. 3 shows a further exemplary embodiment of a light distribution element 1 including holder 5. The light distribution element 1 is also a circular disc here, however, the opening for the passage of a catheter is designed as a slot 40. The slot 40 extends in this case from an edge region of the light distribution element 1 to its center point. Due to this design of the passage opening as a slot 40, the light distribution element 1 or the entire illumination device can also be added or removed after the placement of the catheter.

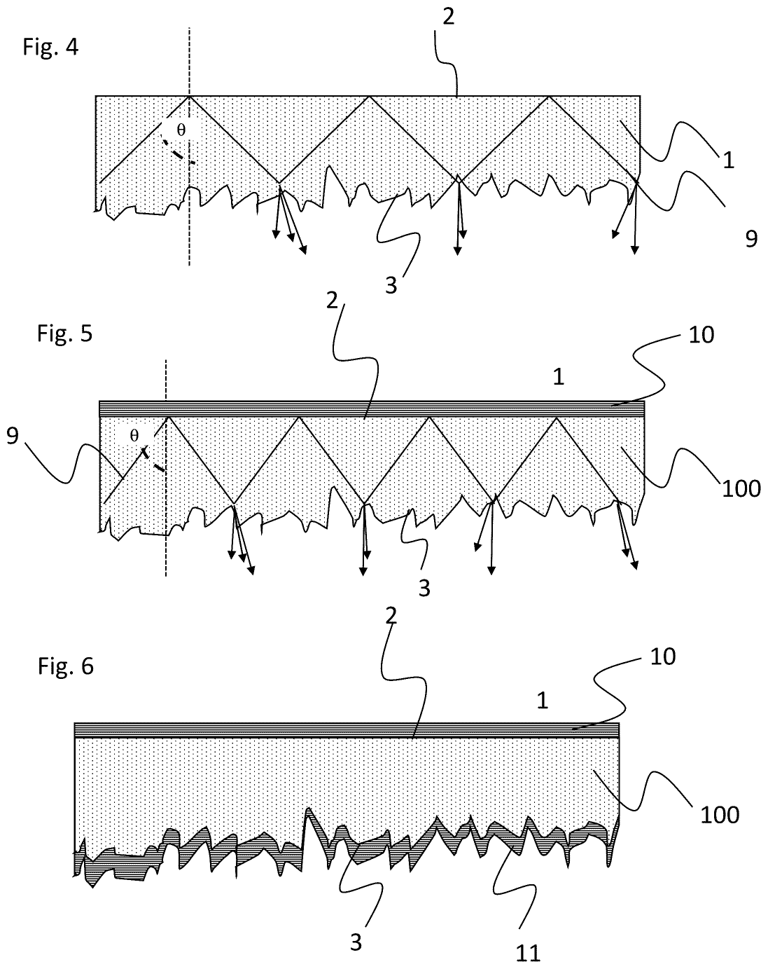

Cross sections through the light distribution element 1 of various exemplary embodiments are schematically shown in FIGS. 4 to 6. A one-piece light distribution element 1 is shown in FIG. 4, which has a smooth lateral surface 2 and a structured lateral surface 3. The lateral surface 3 has a higher roughness RMS than the lateral surface 2 opposite thereto and is the decoupling lateral surface of the light distribution element. The line 9 symbolizes the beam path of the light coupled in, the decoupled light is shown by the arrows. Since the material of the light distribution element 1 is transparent for the light coupled in, it propagates within the light distribution element 1. If the material of the light distribution element has the index of refraction n, light beams having the angle of incidence θ are totally reflected within the light distribution element at the lateral surfaces if the angles of incidence are greater than the critical angle θ_{c}=arcsin (1/n). The light 9 is thus reflected on the lateral surface 2 of the light distribution element 1 and is incident on the roughened lateral surface 3. Complete reflection of the light 9 back into the interior of the light distribution element 1 does not take place due to the rough structure of the lateral surface 3, but rather a part of the light is decoupled from the lateral surface 3 and scattered at the same time. The roughness or grain of the roughened lateral surface is preferably set so that the light is predominantly scattered forward and thus leaves the light distribution element 1 through the decoupling lateral surface 2. The light distribution element 1 therefore also functions as a diffuser.

FIG. 5 shows a further embodiment of a light distribution element 1. The light distribution element 1 comprises here, in addition to a substrate 100 made of a material transparent to the light 9, a reflective coating 10. The reflective coating 10 is applied to the lateral surface 2, i.e. to the lateral surface which does not decouple. The reflection of the light within the light distribution element 1 can be improved by the coating 10 and light losses can thus be minimized. Depending on the quality or condition of the coating 10, in contrast to the embodiment in FIG. 4, the reflection can also take place for light beams in which the angle of incidence is θ<θ_{c}. The coating 10 can be formed as a single-layer or multilayer coating and can comprise, for example, silver layers or aluminum layers. In the exemplary embodiment shown in FIG. 5, the decoupling lateral surface 3 is the lateral surface having the greater roughness. However, it is also possible to form the lateral surface 3 as a smooth surface and the opposite lateral surface 2 as a rough lateral surface. The substrate 100 can be amorphous or crystalline. According to one embodiment, the substrate 100 is formed by quartz glass, sapphire, or crystalline CaF₂ or crystalline MgF₂. The use of water-enriched quartz glass as a material for the substrate 100 has proven to be particularly advantageous. Water-enriched quartz or quartz glass thus has a reduced damping tendency in relation to conventional quartz. The tendency toward polarization of the water-enriched quartz glass is also significantly lower.

FIG. 6 shows a further embodiment of the light distribution element 1. The substrate 100 has coatings on both lateral surfaces 2, 3 here. Analogously to the exemplary embodiment shown in FIG. 5, a reflective coating 10 is located on the lateral surface 2. The decoupling lateral surface 3 is structured as in the example shown in FIG. 5, but additionally has a partially-reflective coating 11. The proportion of the decoupled light can be influenced by the partially-reflective coating 11.

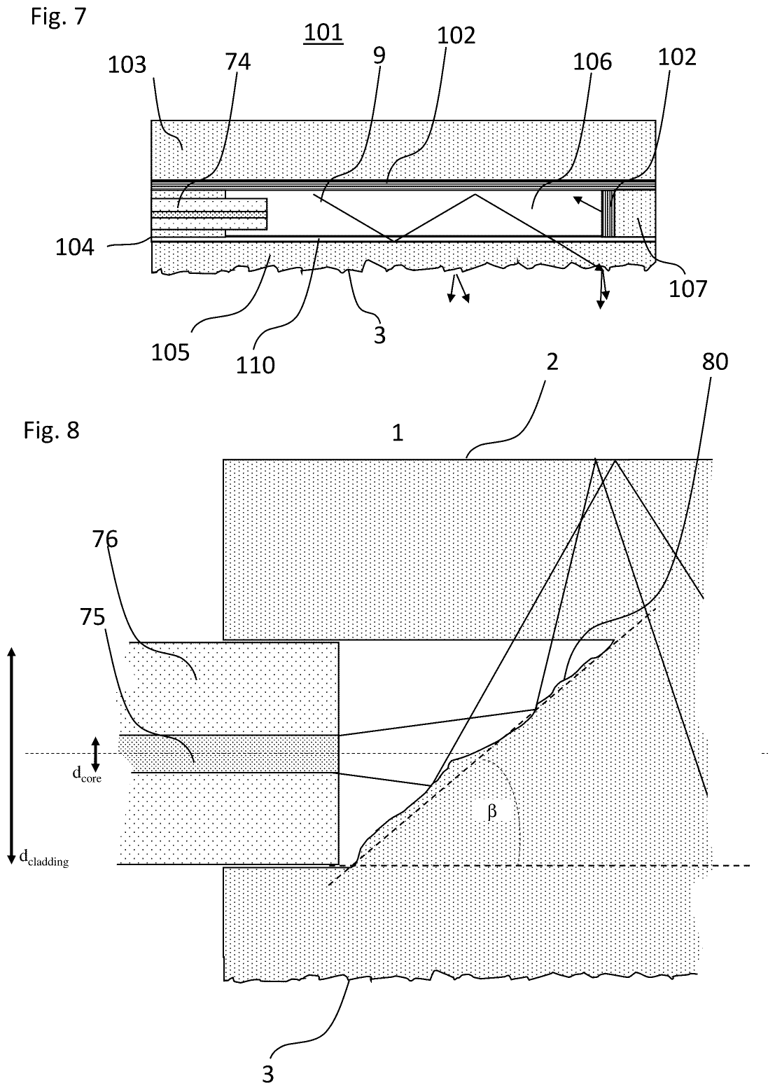

FIG. 7 shows a schematic cross section through a light distribution element 101. The light distribution element 101 comprises multiple substrate components 103, 104, 105, and 107. Component 105 and preferably the components 103, 104, and 107 consist of a material transparent to the light coupled in. The decoupling lateral surface 3 is formed by the substrate component 105. The substrate components 103, 104, 105, and 107 are arranged so that a cavity 106 is formed between them, which is filled with air or a gas or possibly is even evacuated. A light guide 74 is guided into the cavity 106, where the light 9 is emitted, through an opening in the component 104. The cavity 106 is delimited by surfaces of the components 104, 105, and 107. The component 107 is located opposite to the component 104, the components 103 and 105 are located above the component 104. The components 104 and 107 can also be formed as one component, i.e. in one piece. In this embodiment, this component encloses the cavity 106. The surfaces of the components 103 and 107 which delimit the cavity 106 are provided with reflective coatings 102. The surface of the component 105 which delimits the cavity 106 has a partially-reflective coating 110. The light emerges from the light guide 74 into the cavity 106 and propagates. An exemplary beam path is shown by the line 9. If the light is incident on the reflective coatings 102, it is guided back into the cavity 106. The coating 110 deposited on the component 105 is a partially-reflective coating, a part of the light is therefore coupled into the component 105 through the coating 110. The coupled-in part of the light propagates within the component 105 and is decoupled and scattered at its structured lateral surface 3. The proportion of the light which is decoupled through the component 105 can be set via the coating 110 or its transmittance. A homogeneous light intensity can be obtained here over the entire decoupling surface 3 by repeated reflection within the cavity 106.

The embodiment shown in FIG. 7 is suitable in particular for applications in which the light is to be coupled laterally into the light distribution element 1, 101. Due to the more complex structure and selection of the coatings of the components 103, 104, 105, and 107, particularly homogeneously distributed light intensities can be obtained. It is also conceivable that one of the components 103 and/or 105, contrary to what is shown here, can assume a geometry other than a planar geometry, thus, for example, can be designed as a segment of a sphere, such as a so-called hourglass, or in general having a 3D-shaped surface. It is therefore also possible to intervene in the light distribution or homogenization of the light intensities. Furthermore, the light distribution elements 101 according to the figures shown up to this point can also be covered and/or enclosed by further components, which are not transparent to UV light, on the side facing away from the treatment side. This is done, for example, to suppress emission of UV light outward away from the treatment side.

FIG. 8 shows a schematic cross section of a light distribution element 1 having inclined coupling surface 80. The coupling surface 80 has an angle β to the horizontal in the range of 10° to 80°, preferably in the range of 30° to 50°. The light guide 75 is bevelled or spliced at the distal end, so that the light beam emerging from the light guide 75 is already expanded. The light is additionally homogeneously distributed in the light distribution element 1 by reflection of the light at the bevelled coupling surface 80 before it emerges through the decoupling lateral surface 3 and is scattered at the rough or structured surface. The homogenizing effect of the light distribution element 1 can be further improved in that the surface of the coupling surface 80 is also structured or roughened and thus further expands the original light cone originating from the fiber by scattering or diffraction.

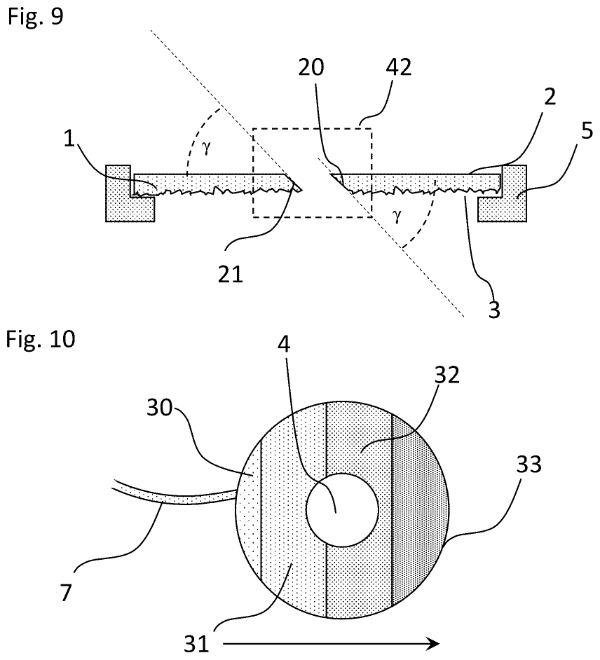

FIG. 9 shows a further exemplary embodiment of the light distribution element 1. In this embodiment, the passage opening 42 does not extend perpendicularly, but rather obliquely through the light distribution element 1. The passage opening 42 therefore has inclined walls 20, 21. The walls form an angle γ, which is preferably in the range of 5 to 50°, with the lateral surfaces 2, 3 of the light distribution element 1. Due to the inclined passage opening 42, the illumination device is particularly well suitable for use with catheters which are placed at a flat angle, since the catheters are guided through the hole at a defined angle in relation to the skin surface. In the exemplary embodiment shown in FIG. 9, the passage opening 42 is arranged centrally within the light distribution element 1. Alternatively, however, it is also possible to place the passage opening 42 off-center depending on the angle γ.

FIG. 10 shows a schematic view of the decoupling lateral surface of a further embodiment of a light distribution element. The decoupling lateral surface is subdivided by way of example into the four sections 30, 31, 32, and 33. The light is coupled via the light guide 7 at the end side of the light distribution element (not shown) into the area of the section 30. The sections 30, 31, 32, 33 differ with respect to their roughness RMS, wherein the roughness increases from the section 30 to the section 33. This course of the roughness is symbolized by the arrow. The roughness increases with increasing distance from the coupling point. In this way, the proportion of the light which is decoupled through the decoupling lateral surface of the light distribution element is lower in regions close to the coupling point than in regions farther away from the coupling point. Due to the increase of the roughness and the increase of the degree of decoupling connected thereto with increasing distance from the coupling point, a loss of the light intensity within the light distribution element 1 is therefore compensated for. This ensures a homogeneous light intensity of the decoupled light over the entire decoupling lateral surface 3. The local change of the roughness of the lateral surface 2 can advantageously take place in more than four sections and particularly preferably gradually for homogeneous light distribution. According to another embodiment, the roughness thus increases continuously from the section 30 to the section 33. Depending on the embodiment having one or also multiple light guides 7 for coupling in light, a roughness change other than the linear one shown here, which is possibly gradual, can also be advantageous or necessary. The roughness change can also be made circular or can be designed as concentric to or around the passage opening 4.

The roughness should therefore increase with increasing distance from the coupling point and the outcoupling of light from the light distribution element should be promoted or varied with increasing distance from the coupling point. This increase or variation can be designed in steps or discrete consecutive areas, each with constant roughness, as well as a gradient, i.e. a continuous change in roughness, as a roughness profile. Both the steps and a gradient can be linear, i.e. the roughness increases linearly from the coupling point. Similarly, the change, in particular the increase in roughness, can also be designed to follow an exponential function, i.e. an exponential progression or variation, or another function, in order to achieve optimal or special illumination. In particular, in the case of the in-coupling of light at several points of the light distribution element 1 via two or more light guides 7, a superposition roughness profile is obtained, as it were, by the superposition of the individual roughness profiles, each starting from an in-coupling point. In the case of a circular light distribution element 1 with several radially uniformly arranged coupling points, this superposition roughness profile will approach a circular or concentric course with an increasing number of coupling points. Depending on the geometry of the light distribution element 1, the number of coupling points and the required illumination complex stepped or graduated roughness or superposition roughness profiles can result or be set.

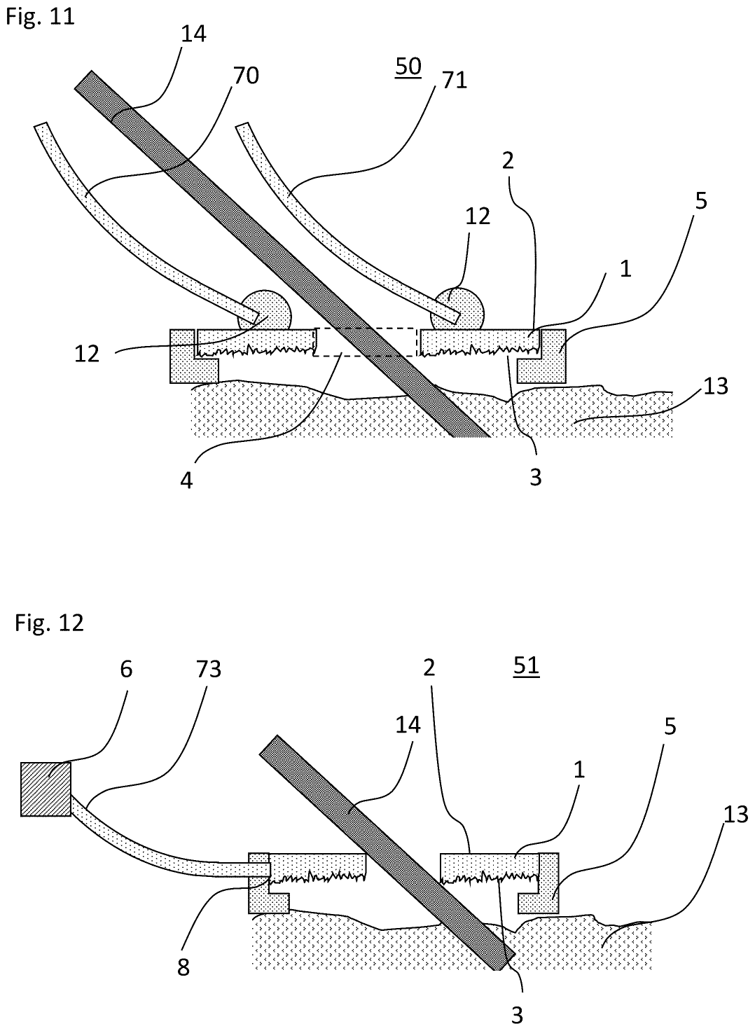

FIG. 11 shows a device for sterilization of the skin 50 according to an exemplary embodiment in cross section. The device 50 comprises an illumination device having a light distribution element 1, a holder 5, light guides 70, 71, a light source (not shown), and a catheter 14. The light distribution element 1 is mounted on a holding device 5, wherein the holding device 5 has an L-shaped profile and extends beyond the light distribution element 1 so that it functions as a spacer between the light distribution element 1 and the skin 13. In this case, the holding device 5 has skin contact at least at points. In this embodiment, the holding device is manufactured from an organic polymer, preferably from a polysiloxane or a polycarbonate. The light coupling takes place via the lateral surface 2 of the light distribution element 1 which is opposite to the decoupling lateral surface 3. The light guides 70, 71 are materially bonded by a glass solder or an adhesive 12 to the light distribution element 1. The passage opening 4 is arranged in the center of the light distribution element 1. A catheter 14 is guided through the passage opening 4. The light distribution element 1 is aligned here so that the decoupling lateral surface 3 is directed toward the skin surface 13. The region 13 of the skin surface which is located below the light distribution element 1 is therefore irradiated with the decoupled light and thus disinfected or sterilized. An illumination field having homogeneous or at least substantially homogeneous light intensity is generated by the scattering of the decoupled light on the rough lateral surface 3 here.

FIG. 12 shows a further exemplary embodiment of a device for sterilizing the skin 51. The structure of the device 51 corresponds in large parts to the structure of the exemplary embodiment 50 shown in FIG. 11. Notwithstanding this, the coupling in of the light through the light guide 73 takes place via the end side 8 or a section of the circumferential edge which forms the end side 8, however.

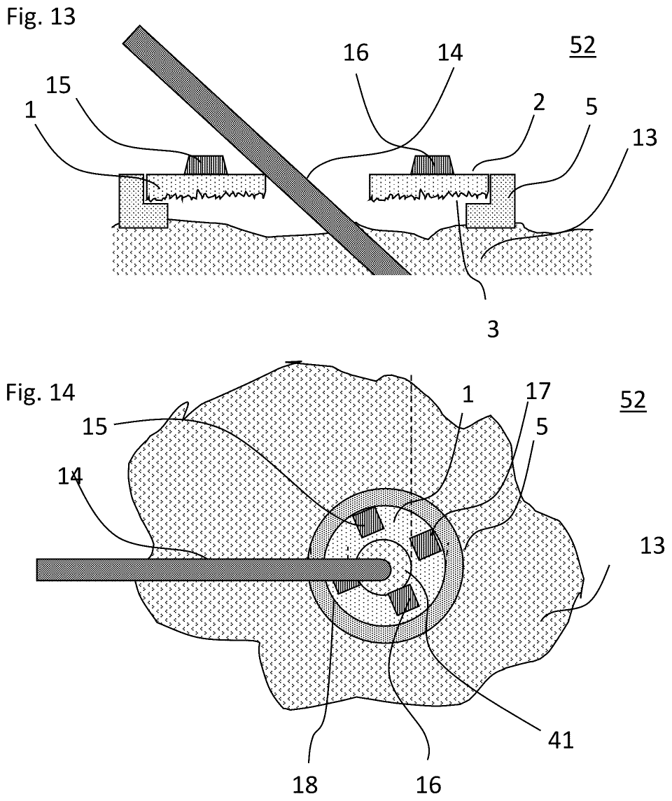

A further exemplary embodiment of a device for sterilizing the skin 52 is schematically shown in FIG. 13, wherein FIG. 13 shows a schematic cross section of the exemplary embodiment and FIG. 14 shows a schematic illustration in a top view. In contrast to the exemplary embodiments 50, 51 shown in FIGS. 11 and 12, in this exemplary embodiment the light sources 15, 16, 17, 18 are arranged on the lateral surface 2 of the light distribution element 1. The light sources 15, 16, 17, 18 are preferably UV LEDs which emit UV light in the wavelength range of 250 to 300 nm. The light sources 15, 16, 17, 18 can be adhesively bonded to the lateral surface 2 of the light distribution element 1 or also held by a device (not shown). The light emitted by the light sources 15, 16, 17, 18 is therefore coupled directly into the light distribution element 1. In the exemplary embodiment 52 shown, the device has four light sources 15, 16, 17, 18, which are each arranged at the same distance from the center point of the light distribution element 1 and have equidistant distances from one another. Due to the use of multiple light sources 15, 16, 17, 18, the coupling in of the light already takes place uniformly distributed over the light distribution element 1, which also has an effect on the homogeneity of the decoupled light intensity.

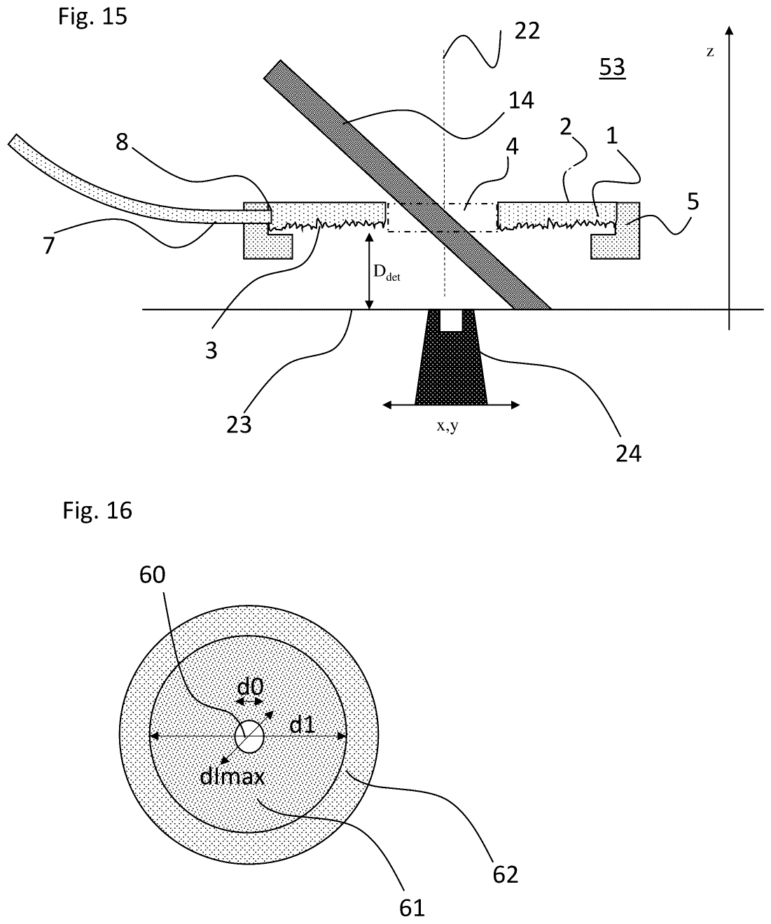

FIG. 15 shows a schematic illustration of a measuring method for determining the homogeneity of the light emitted by the light distribution element. A detector 24 is moved along the detection plane 23 and starting from the center point 22 of the passage opening 4 below the device for sterilizing the skin 53. The light intensity is measured continuously in this case, so that a location-resolved light intensity, i.e. the light intensity as a function of the distance from the center point 22 is obtained. The detector 24 comprises a photodiode 24 as a sensor. A silicon photodiode, for example, a photodiode of the type Thorlabs FDS010 can be used as the photodiode. The detector is calibrated for a wavelength range between 200 nm and 290 nm and can detect energies below 1 mW with a resolution of at least 50 μW. For power calibration of the photodiode, for example, calibrated detectors of the type MKS Ophir, PD300R-UV or Coherent Laser PowerMax-USB PS10 can be used.

The detector 24 is moved along the detection plane 23 in the x and y direction over at least a range of 20 mm and the light energy is measured with a spatial resolution of at least 50 μm. The detection plane 23 has a distance to the decoupling lateral surface of the light distribution element of D_{det}=10 mm. The measured light energies are divided by the detection area of the photodiode to ascertain the light intensity.

FIG. 16 schematically shows a top view of a surface 62 illuminated by a device according to the invention for sterilizing the skin. The surface 62 corresponds to the surface of the puncture point of the catheter and has a diameter do. The diameter do corresponds in this case to the diameter of the catheter. The surface 61 corresponds to a region measured using the device shown in FIG. 15 and has a diameter d1.

One exemplary embodiment of the device provides here that the irradiation intensity of the UV light decoupled from the light distribution element on a surface 62 which is arranged below the light distribution element is homogenized so that along a circular delimitation line d₁ at a selectable distance from the center point of the opening to at most 2 cm from this center point, the ratio of the maximum of the irradiation intensity and the minimum of the irradiation intensity has a ratio of at most 3, preferably of at most 2. The following therefore applies:

I_{max,d1}/I_{min,d1}≤3, preferably ≤2 and particularly preferably ≤1 with d₁<4 cm.

Moreover, the irradiated surface 62 has its intensity maximum at a distance d_{Imax}, wherein for d_{Imax}: d_{Imax}≤3 cm, preferably ≤2 cm. the region 63 having the maximum light intensity is therefore at a distance of at most 1.5 cm, preferably at most 2 cm from the center point of the puncture point having the diameter do.

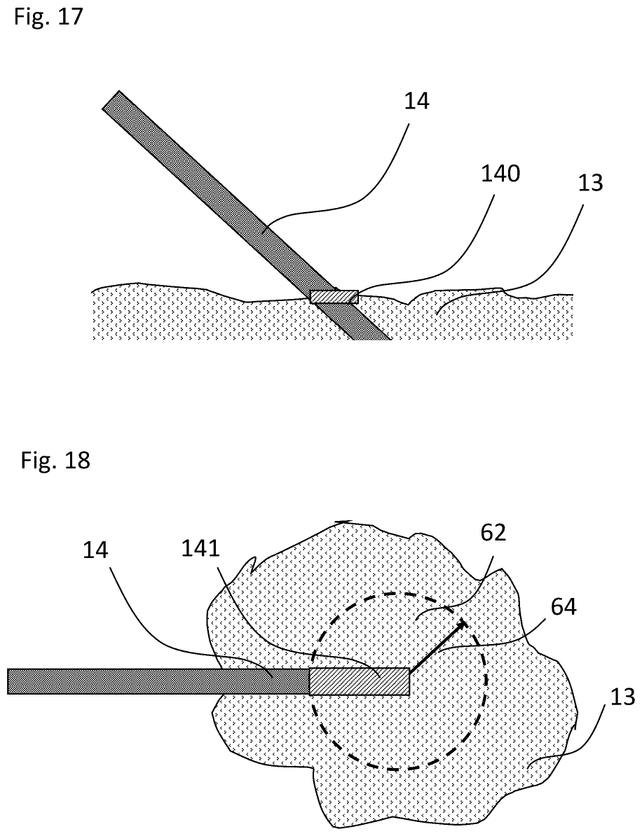

FIGS. 17 and 18 schematically show the regions of minimum shading 140 and maximum shading 141 within the illuminated surface 62 by the catheter 14. FIG. 17 is a schematic top view, FIG. 18 is a schematic side view. The region 140, which is always shaded by the catheter independently of the arrangement of the illumination device, is the puncture point of the catheter. The area of the region 140 therefore corresponds to the area of the puncture point or the cross-sectional area of the catheter and results as follows:

$$1. {A}_{in} = π * {d}_{c}^{2} / ( 2 * cos α )$$

with d_{c}=catheter diameter and a the puncture angle of the catheter. The device therefore necessarily has a proportion of shaded area Sₘᵢₙ, which can be calculated as follows:

$$ii . {S}_{min} = {A}_{in} / A = {d}_{c}^{2} / ( 4 {R}^{2} * cos α )$$

with A=illuminated area, R=radius of the illuminated area.

In a device having an illuminated area having a diameter of 15 mm and a catheter diameter of 2.1 mm, a minimum shading proportion therefore results of Sₘᵢₙ=2%.

The maximum region A_{c} which can be shaded by the catheter is identified by the reference sign 141 and can be calculated as follows:

A_{c}=R*d_{c} i.
with R=radius of the illuminated area and d_{c}=catheter diameter.

The following accordingly applies for the maximum shading proportion Sₘₐₓ

$$i . {S}_{max} = {A}_{c} / A = {d}_{c} / ( π * R )$$

With the above-mentioned dimensions dc=2.1 mm and R=7.5 mm, a theoretical maximum shaded proportion Sₘₐₓ of 9% therefore results.

However, this proportion can be significantly reduced by the use of the light distribution element in the devices according to the invention. According to one embodiment, the following therefore applies for the proportion of the shaded regions Sᵣₑₐₗ:

Sₘᵢₙ<Sᵣₑₐₗ≤0.7*Sₘₐₓ, preferably Sₘᵢₙ<Sᵣₑₐₗ≤0.5*Sₘₐₓ, and particularly preferably Sₘᵢₙ<Sᵣₑₐₗ≤0.3*Sₘₐₓ.

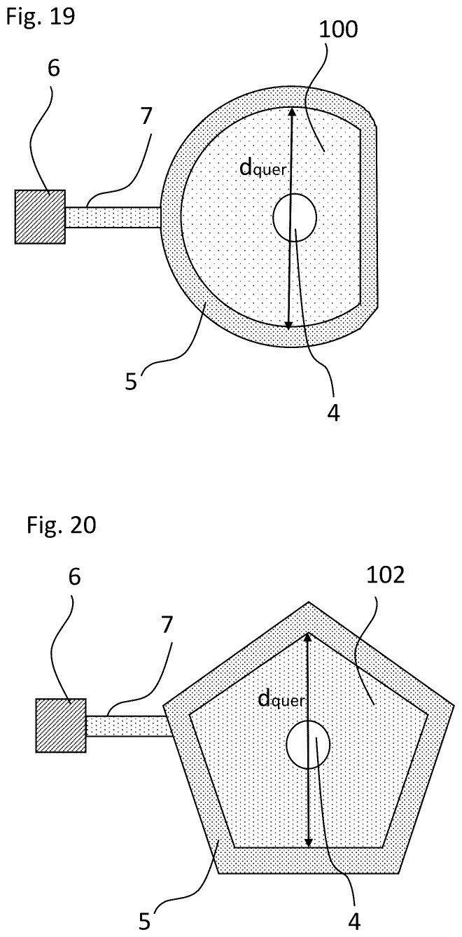

FIGS. 19 and 20 schematically show further embodiments of the illumination device in a top view. FIG. 19 shows an illumination device for this purpose in which the light distribution element 100 does not form a complete circle, but rather is D-shaped. The arrow shows the maximum transverse dimension d_{quer}. The light distribution element 102 shown in FIG. 20, in contrast, has a pentagonal, i.e. polygonal light distribution element.

### LIST OF REFERENCE NUMERALS

| | |
|---|---|
| 1, 100, 101, 102 | light distribution element |
| 2 | lateral surface of the light distribution |
| | element without light decoupling |
| 3 | decoupling lateral surface of the light |
| | distribution element |
| 4, 40, 41 | passage opening |
| 5 | holder element |
| 6 | light source |
| 7, 70, 71, 73, | light guide |
| 74, 75 | |
| 8 | end side of the light distribution |
| | element 1 |
| 9 | light beam |
| 10, 102 | reflective coating |
| 11, 110 | partially reflective coating |
| 12 | glass solder |
| 13 | skin |
| 14 | catheter |
| 15, 16, 17, 18 | UV-LED |
| 20, 21 | walls of the passage opening |
| 22 | center point of the light distribution |
| | element 1 |
| 23 | detection plane |
| 24 | movable photodiode |
| 30, 31, 32, 33 | sections of the lateral surface 3 |
| 50, 51, 52, 53 | device for sterilizing the skin |
| 60 | puncture point |
| 61 | illuminated surface |
| 62 | region of 61 in which the light intensity |
| | is ascertained |
| 63 | region of 61 having the maximum light |
| | intensity |
| 64 | detector |
| 76 | cladding of the light guide 75 |
| 103, 104, 105, | components of 101 |
| 1075, and 107 | |
| 106 | air |
| 140 | minimum shading region |
| 141 | maximum shading region |

1. An illumination device comprising a light source, which emits light at a wavelength in the range of 180 nm to 360 nm, and a light distribution element having two opposing lateral surfaces, wherein the light distribution element comprises a material transparent or at least largely transparent to the light coupled in, wherein the light of the light source is coupled into the light distribution element and emerges from at least one of the two lateral surfaces of the light distribution element, wherein the light distribution element has structures for scattering the light coupled in, in order to at least partially deflect the light so that it emerges from at least one of the lateral surfaces, wherein the light distribution element has a passage opening which extends from one lateral surface of the light distribution element to the other lateral surface.

2. The illumination device according to claim 1, wherein the illumination device is suitable for use in a medical treatment system and preferably the passage opening is designed as a passage for a catheter.

3. The illumination device according to claim 1, wherein a region, which is irradiated by the light emerging from the decoupling lateral surface of the light distribution element, has an area in the range of 1 cm² to 25 cm².

4. The illumination device according to claim 1, wherein the light distribution element is shaped circular, ellipsoidal, or D-shaped and preferably has a maximum transverse dimension in the range of 1 to 8 cm.

5. The illumination device according to claim 1, wherein the light source emits UV light of a wavelength in the range of 180 to 250 nm.

6. The illumination device according to claim 1, wherein the light distribution element comprises a material, the damping of which in the wavelength range of the light coupled in is less than −3 dB/cm, and/or the light distribution element comprises amorphous materials or a crystalline material.

7. The illumination device according to claim 1, wherein at least one lateral surface of the light distribution element has at least in sections a roughness RMS in the range of 1 to 400 nm.

8. The illumination device according to claim 7, wherein the decoupling lateral surface of the light distribution element has locally differing roughness.

9. The illumination device according to claim 1, wherein at least one lateral surface of the light distribution element has microstructuring for setting the local intensity of the decoupled light.

10. The illumination device according to claim 1, wherein one of the two lateral surfaces of the light distribution element has a higher roughness than the opposing lateral surface.

11. The illumination device according to claim 1, wherein at least one of the lateral surfaces of the light distribution element has a reflective coating.

12. The illumination device according to claim 1, wherein the light distribution element is designed as a circular disc having a diameter in the range of 20 to 30 mm and a thickness in the range of 0.4 to 1.5 mm, wherein the passage opening is arranged in the center of the disc and the passage opening has a diameter in the range of 2 to 6 mm.

13. The illumination device according to claim 1, wherein the light distribution element is fixed in a circumferential holder element, wherein a region of the holder element protrudes beyond the decoupling lateral surface of the light distribution element.

14. The illumination device according to claim 13, wherein the holder element comprises an organic polymer.

15. The illumination device according to claim 1, wherein the light of the light source is coupled via at least one light guide into the light distribution element.

16. The illumination device according to claim 15, wherein the light guide is spliced or adhesively bonded to the light distribution element.

17. The illumination device according to claim 15, wherein the light guide at the distal end, which is connected to the light distribution element, has a numeric aperture NA>0.1.

18. The illumination device according to claim 15, wherein the light guide is connected to the light distribution element via the lateral surface of the light distribution element, which is opposite to the decoupling lateral surface.

19. The illumination device according to claim 15, wherein the light guide is connected to the light distribution element via an end side of the light distribution element.

20. The illumination device according to claim 19, wherein the light is coupled in via an inclined surface of the light distribution element.

21. The illumination device according to claim 20, wherein the inclined coupling-in surface has a roughness RMS of >1 nm.

22. The illumination device according to claim 1, wherein the light is coupled in via the lateral surface of the light distribution element, which is opposite to the decoupling lateral surface, and the light source is connected in a formfitting or materially-bonded manner to this lateral surface.

23. The illumination device according to claim 22, wherein the illumination device has at least one light source and the light sources are preferably UV LEDs.

24. A device for sterilizing the skin, comprising an illumination device according to claim 1 and a catheter, wherein the catheter is guided through the passage opening so that the end of the catheter, using which the catheter is introduced through the skin into the patient, is located on the side of the light distribution element having the decoupling lateral surface and wherein the illumination device comprises a circumferential holder element, which protrudes beyond the light distribution element at least on the decoupling lateral surface of the light distribution element and forms a spacer between the skin and the light distribution element.
